(19) United States
(12) Patent Application Publication
(10) Pub. No.: US 2015/0050291 A1
(43) Pub. Date: Feb. 19, 2015

(54) DETECTION AND MODULATION OF RHEUMATOID ARTHRITIS

(71) Applicant: UNIVERSITYOF MANITOBA, Wininpeg, (CA)

(72) Inventor: Neelhoffer Mookerjee, Winnipeg (CA)

(73) Assignee: UNIVERSITYOF MANITOBA, Winnipeg, Manitoba (CA)

(21) Appl. No.: 14/386,597

(22) PCT Filed: Mar. 19, 2013
(86) PCT No.: PCT/CA2013/000265

§ 371 Date: Sep. 19, 2014

Related U.S. Application Data
(60) Provisional application No. 61/612,593, filed on Mar. 19, 2012.
(60) Provisional application No. 61/614,425, filed on Mar. 22, 2012.

(51) Int. Cl. G01N 33/68; C07K 16/24

(52) U.S. Cl. CPC G01N 33/6803; C07K 16/24; G01N 2800/102
USPC 424/158.1; 435/7.92; 530/389.2; 435/6.12

(57) ABSTRACT

A method for detecting and/or monitoring a chronic inflammation condition associated with rheumatoid arthritis. The method comprises the steps of: (i) collecting a sample; (ii) detecting in the sample, one or more of a HMGN1 protein, a LCP-1 protein, a prtn3 protein, a sec22b protein, and a PYGL glycogen phosphorylase protein thereby producing a result; and (iii) correlating the result with a control. A test kit for diagnosing or monitoring a chronic inflammatory condition associated with rheumatoid arthritis. The test kit comprises an antibody for complexing with one of a HMGN1 protein, a LCP-1 protein, a prtn3 protein, a sec22b protein, and a PYGL glycogen phosphorylase protein. A composition for treatment of a chronic inflammation condition associated with rheumatoid arthritis, comprising a therapeutically effective amount of an exogenous agonist for decreasing cellular production of one or more of a HMGN1 protein, a LCP-1 protein, a prtn3 protein, a sec22b protein, and a PYGL glycogen phosphorylase protein.

### TECHNICAL FIELD

The present invention relates to methods for diagnosing and/or monitoring rheumatoid arthritis. The invention also relates to therapeutic targets for modulation of rheumatoid arthritis, to therapeutic compositions directed to the targets, and to use of the compositions for modulation of rheumatoid arthritis.

### BACKGROUND

A wide range of diseases are characterized by chronic inflammation which include rheumatoid arthritis (RA), inflammatory bowel disease, atherosclerosis, and certain cancers. Arthritis and rheumatism affect nearly 15% of the North American population and are associated with significant economic burdens. These are complex diseases which involve many cells, molecules, and processes, therefore represent a major challenge for the development of effective therapies. Two critical pro-inflammatory cytokines well established in the pathogenesis of chronic inflammatory diseases including RA are TNF-α and IL-1β. The cellular cross-talk and downstream responses induced by these cytokines are not completely delineated.

Molecular indicators of a disease state or activity (biomarkers) can provide insight into the pathogenesis, new therapeutic targets, and molecules that may be used to predict the responsiveness of candidate therapeutics. Identification of such molecular indicators is an important goal in biomedical research, including for diseases characterized by chronic inflammation. Gene-expression monitoring (GEM) or cytokine profiling from serum or other body fluids to define biomarkers for systemic inflammation has been used in recent years. However, GEM is not a robust indicator for the phenotype of the disease, and multianalyte cytokine profiling are hypothesis-driven surrogate endpoints that does not differentiate between different systemic inflammatory disorders³. Mass spectrometry (MS)-based differential proteomics approaches has provided information regarding alterations in global protein profiles contributing to diseased phenotypic state, thus aiding in the diagnosis, prognosis and response-prediction to therapeutics in various clinical conditions. Also, global proteomic approaches such as 2D gel electrophoresis or with isotope labelling does not differentiate between newly synthesized (nascent) proteins in response to a stimulus and those from the pre-existing pools, since they are chemically identical.

### SUMMARY

Exemplary embodiments of the present invention relate to methods for diagnosing or monitoring a chronic inflammation condition associated with rheumatoid arthritis, to test kits for diagnosing or monitoring a chronic inflammation condition associated with rheumatoid arthritis, and to compositions for treatment of a chronic inflammation condition associated with rheumatoid arthritis.

An exemplary embodiment of the present invention relates to a method comprising the steps of collecting a biological sample from a subject, detecting in the biological sample, one or more of a HMGN1 protein, a LCP-1 protein, a prtn3 protein, a sec22b protein, and a PYGL glycogen phosphorylase protein to produce a test result, and correlating the test result with a control reference standard.

Another exemplary embodiment of the present invention relates to test kits for diagnosing or monitoring a chronic inflammatory condition associated with rheumatoid arthritis in a test sample. The exemplary test kits comprise one or more antibodies selected for complexing with one or more of a HMGN1 protein, a LCP-1 protein, a prtn3 protein, a sec22b protein, and a PYGL glycogen phosphorylase protein.

Another exemplary embodiment of the present invention relates to compositions for treatment of a chronic inflammation condition associated with rheumatoid arthritis. The compositions comprise a therapeutically effective amount of one or more exogenous agonists for decreasing cellular production of and/or cellular activation of and/or cellular expression one or more of a HMGN1 protein, a LCP-1 protein, a prtn3 protein, a sec22b protein, and a PYGL glycogen phosphorylase protein.

### BRIEF DESCRIPTION OF THE DRAWINGS

The present invention will be described in conjunction with reference to the following drawings, in which:

FIG. 1(A) is a chart showing the effects of stimulating AHA-labelled human monocytic THP-1 cells with either TNF-α or IL-1β (10 ng/ml) for 4 hr on IL-1β production, and 1(B) is representative gel showing the presence of AHA-containing nascent proteins produced by human monocytic THP-1 cells stimulated with either TNF-α or IL-1β; and

FIGS. 2(A) and 2(C) are charts showing the effects of TNF-α on RNA production (A) and gene expression (C) by human macrophage-like THP-1 cells, while 2(B) and 2(D) are charts showing the effects of IL-1β on RNA production (B) and gene expression (D) by human macrophage-like THP-1 cells.

### DETAILED DESCRIPTION

Two pro-inflammatory cytokines well-defined in the pathogenesis of rheumatoid arthritis (RA) and other chronic inflammatory disorders are Tumour necrosis factor-alpha (TNF-α) and Interleukin-1β (IL-1β). These cytokines contribute to complex cellular processes in inflammatory diseases. Many therapies target these cytokines for controlling inflammation, but responses to these therapies are variable among patients. An impediment in the development of new therapeutic strategies for chronic inflammatory diseases is the limited understanding of underlying molecular mechanisms induced in the presence of these inflammatory stimuli. Therefore, the objective of this study was to identify newly synthesized (nascent) proteins induced by TNF-α and IL-1β. We hypothesized that identification of de novo (nascent) proteins that are induced specifically in the presence of either TNF-α or IL-1β may help to delineate cellular adaptations in presence of these inflammatory stimuli, and lead to the identification of new molecular indicators that could be further developed as therapeutic targets. Of specific advantage would be identification of common proteins that are actively induced in the presence of both TNF-α and IL-1β, these proteins may serve as alternative therapeutic targets, especially important for non-responders to existing therapies.

In this study, we optimized an experimental approach using a combination of metabolic labelling and quantitative proteomics to identify and quantitative nascent proteins in the presence of exogenous stimuli as follows; we used bioorthogonal non-canonical amino acid tagging (BONCAT) with a surrogate for methionine, L-azidohomolanine (AHA), to label cytokine-induced nascent proteins. The AHA-containing nascent proteins were further tagged with alkyne-biotin for enrichment using affinity purification. Subsequently the nascent proteins were quantified using isobaric iTRAQ® reagents (iTRAQ is a registered trademark of AB Sciex Pte. Ltd., Singapore) and mass spectrometry (MS). We identified 16 nascent proteins that were significantly (p≦0.05) induced upon cytokine stimulation after 4 hr of stimulation. To validate the identified protein candidates we monitored the kinetics of transcriptional responses after 1, 2 and 4 hr of cytokine stimulation employing quantitative real-time PCR (qRT-PCR). Of the 16 candidates monitored by qRT-PCR, there were five common candidates demonstrated to be induced at the transcriptional level upon stimulation with either TNF-α or IL-1β. Four of these candidates showed robust (more than 4-fold up-regulation of gene expression) and significant (p≦0.05) response; (i) a nuclesome binding protein HMGN1, (ii) lymphocyte cytosolic protein 1 (LCP-1), (iii) a serine protease, prtn3, also known as C-ANCA antigen, and (iv) a vesicle trafficking protein sec22b. A glycogen phosphorylase, PYGL, which is an important metabolic enzyme, was modestly (≧1.5-fold, p≦0.01) induced by both TNF-α and IL-1β. Gene expression of two molecular candidates, (i) a RAS family oncogene, RAB14, and (ii) Eifa3, were modestly (≧1.5-fold) but significantly (p≦0.01) up-regulated upon stimulation with IL-1β but not TNF-α. This is the first study to employ a combination of BONCAT and iTRAQ® proteomics approaches to quantitatively define nascent proteins induced upon stimulation with either TNF-α or IL-1β.

### EXAMPLES

### Example 1

### Materials and Methods

Cell culture: Human monocytic THP-1 (ATCC® TIB-202) cells (ATCC is a registered trademark of the American Type Culture Collection Corp., Manassas, Va., USA) and Jurkat T-cell line (ATCC® TIB-152) were cultured in RPMI-1640 media containing 2 mM L-glutamine and 1 mM sodium pyruvate, supplemented with 10% (v/v) FBS, and were maintained in a humidified incubator at 37° C. and 5% CO₂. Where indicated, the THP-1 cells were differentiated to plastic-adherent macrophage-like cells by treatment with phorbol 12-myristate 13-acetate (PMA; Sigma-Aldrich Canada Ltd., Oakville ON) as previously described. A rabbit synoviocyte cell line HIG-82 (ATCC® CRL-1832) was cultured in Ham's F-12 growth medium containing glutamine (GIBCO) supplemented with sodium pyruvate (referred to as complete F-12 media henceforth), containing 10% (v/v) FBS in a humidified incubator at 37° C. and 5% CO₂. Confluent HIG-82 cells were trypsinized with 1:3 dilution of 0.5% trypsin-EDTA (Invitrogen Canada Inc., Burlington, ON, Canada) in Hanks' balanced salt solution. Cellular cytotoxicity was evaluated after AHA labelling and upon stimulation with the various cytokines for all the cell types used in this study by monitoring the release of lactate dehydrogenase (LDH) employing a colorimetric detection kit (Roche Diagnostics, Laval, QC, Canada).

Recombinant cytokines: Recombinant human cytokines TNF-α and IL-1β were obtained from eBioscience, Inc. (San Diego, Calif., USA).

ELISA immunoassay: Tissue culture (TC) supernatants were centrifuged at 1500×g for 7 min to obtain cell-free samples, aliquoted and stored at −20° C. until further use. Production of IL-8 was monitored using specific antibody pairs for ELISA (R&D Systems, Inc. Minneapolis, Minn., USA), following the method provided in the manufacturer's instructions. The concentration of IL-8 in the TC supernatants was evaluated by establishing a standard curve with serial dilutions of the recombinant human IL-8 as required.

AHA labelling, biotin tagging of nascent proteins, and affinity purification: Cells were washed with warm D-PBS, and cultured in methionine-free, serum-free RPMI media at 37° C. 5% CO₂ for 60 mM. The cells were then incubated in 100 μM Click-iT® (Click-iT is a registered trademark of Molecular Probes Inc., Eugene, Oreg., USA). AHA (Invitrogen Canada Inc.) in methionine-free and serum-free RPMI media in the presence and absence of either TNF-α (10 ng/ml) or IL-1β (10 ng/ml) for 4 hr. TC supernatants were monitored for the production of IL-8 by ELISA. Metabolic labelling of the cells with AHA reagent in the presence of the cytokine stimulants would result in induced nascent proteins with azide-bearing functional group, thus making them distinct from pool of pre-existing cellular proteins. Also these functional groups were further used to tag the nascent proteins with a biotin-alkyne reagent as follows. The cells were washed with D-PBS, followed by preparing cell lysates in lysis buffer containing 50 mM Tris-HCl, 1 SDS, 250 U/ml of benzonase nuclease and protease and phosphatase inhibitor cocktails (Sigma-Aldrich Canada Ltd.). Total protein was estimated in the cell lysates using micro BCA analysis. Equivalent amount of total protein from each sample was acetone precipitated and then treated with Click-iT® Biotin-alkyne reagent (Invitrogen Canada Inc.) as per the manufacturer's instructions. The biotin-alkyne reagent thus covalently coupled to the reactive azide group of the AHA-modified proteins was used to subsequently enrich the nascent proteins by affinity purification using Ultralink® Immobilized NeutrAvidin® resin (Ultralink and NeutrAvidin are registered trademarks of Pierce Biotechnology Inc., Rockford, Ill., USA), and the bound proteins were eluted using 6 M guanidinium hydrochloride.

Immunoblots: Eluates obtained from affinity purification were electrophoretically resolved on 4-12% NuPAGE® Bis-Tris gels (NuPAGE is a registered trademark of Invitrogen Corp., Carlsbad, Calif., USA), followed by transfer to nitrocellulose membranes (Millipore Canada Inc., Toronto, ON, Canada). The membranes were subsequently blocked with TBST (20 mM Tris pH 7.5, 150 mM NaCl, 0.1% Tween® 20) (Tween is a registered trademark of Uniqema Americas LLC, Wilmington, Del., USA) containing 5% skimmed milk powder, and probed with HRP-linked anti-biotin antibody (Cell Signaling Technology Inc., Boston, Mass., USA) in TBST containing 3% skimmed milk powder. The membranes were developed with the Amersham ECL detection system (GE Healthcare, Baie d'Urfe, QC, Canada) following the manufacturer's instructions.

Quantitative proteomics employing isobaric tag for relative and absolute quantitation (iTRAQ): Amine-modifying iTRAQ® reagents multiplex kit (Applied Biosystems, Foster City, Calif., USA) was employed for relative quantitation of purified nascent proteins. Affinity purified eluates were acetone precipitated at −20° C. overnight. Proteins were dissolved in 20 μl of iTRAQ® dissolution buffer (Applied Biosystems) and further processed as per the manufacturer's instructions. Briefly, proteins were reduced and the cysteines blocked using the reagents in kit, followed by digestion of the protein samples with provided trypsin solution overnight at 37° C. The trypsin-digested protein samples were labelled with the iTRAQ® isobaric tags as follows: Eluates un-stimulated (control) samples was labelled with iTRAQ® isobaric tag 115, TNF-α-stimulated samples with tag 116, and IL-1β-stimulated sample with isobaric tag 117. The contents from each of the iTRAQ® reagent-labelled sample was combined together and processed for nanoflow liquid chromatography coupled to tandem mass spectrometry (LC-MS/MS).

Quantitative real-time PCR (qRT-PCR): RNA was isolated using the Qiagen RNeasy® kit as per the manufacturer's instructions (RNeasy is a registered trademark of Qiagen GmbH Corp., Hilden, Fed. Rep. Germany). Gene expression was subsequently analyzed by qRT-PCR using SuperScript III Platinum Two-Step qRT-PCR Kit with SYBR® Green (SYBR is a registered trademark of Molecular probes Inc., Eugene, Oreg., USA), according to the manufacturer's instructions, in the ABI PRISM 7300 sequence detection system (Applied Biosystems). Fold changes were calculated by the comparative Ct method, after normalization with 18sRNA.

### Results

TNF-α and IL-1β induced differential nascent protein profile. Three different cell types were labelled with AHA: (i) human monocytic THP-1 cell line, (ii) human T-cell Jurkat cell line, and (iii) a rabbit synovial fibroblast HIG-82 cell line. AHA labelling was cytotoxic to the synovial fibroblast cell line as monitored by release of lactate dehydrogenase (LDH) in tissue culture supernatants (data not shown) within 4 hr, but not to either Jurkat or THP-1 cells. Both THP-1 cells and Jurkat cells became plastic adherent after culturing in serum-free, methionine-free media for 60 mins prior to labelling with AHA. Human monocytic THP-1 cells were further used in this study for metabolic labelling with AHA followed by quantitative proteomics using iTRAQ reagents in order to identify nascent proteins induced by inflammatory cytokines.

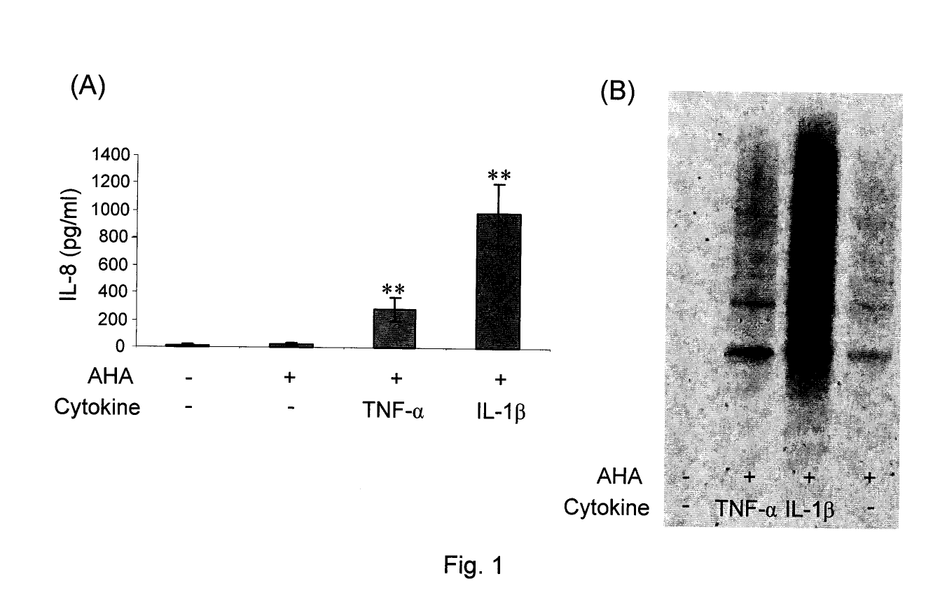

AHA-labelled human monocytic THP-1 cells were stimulated with either IL-1β (10 ng/ml) or TNF-α (10 ng/ml) for 4 hr. Cytokine treatment of AHA-labelled THP-1 cells was not cytotoxic as evaluated by monitoring LDH release in the tissue culture (TC) supernatant (data not shown). TC supernatants were also monitored for IL-8 production by ELISA. Both TNF-α and IL-1β showed significant (p<0.001) production of IL-8 after 4 hr of stimulation (FIG. 1A). IL-8 production was more than 2-fold greater upon IL-1β stimulation when compared to cells stimulated with TNF-α (FIG. 1A). The cell lysates were further treated with a biotin alkyne reagent for tagging biotin onto the azide reactive group of the AHA-containing nascent proteins. The alkyne-biotin tagged AHA-containing nascent proteins were enriched by affinity purification using Neutravidin resin and the eluates were probed with anti-biotin antibody using Western blots. Both IL-1β and TNF-α-treated samples showed increased amounts of biotinylated proteins when compared to un-stimulated control cells (FIG. 1B). The eluates obtained from samples treated with IL-1β appeared to have greater amount of biotinylated enriched proteins compared to samples treated with TNF-α (FIG. 1B), which was consistent with the trend of protein production seen upon monitoring cytokine-induced IL-8 production by ELISA (FIG. 1A).

The AHA-containing nascent proteins enriched by affinity purification were quantitated using isobaric iTRAQ quantitative proteomics reagents. This was done to identify and estimative the relative abundance of nascent proteins enriched in the cytokine-treated samples compared to un-stimulated cells. Protein eluates after affinity purification were labelled with isobaric iTRAQ reagents; tag 115 for un-stimulated control cells, tag 116 for TNF-α-treated cells, and tag 117 for cells treated with IL-1β. Samples from four independent experiments were individually examined by LC-MS/MS. Peptides identified with 95% confidence were selected for further analysis. The mass spectrometry data was analyzed using Global Proteome Machine (GPM-http://www.thegpm.org/). We identified a total of 2440 proteins from the four independent experiments, of which 1449 proteins were identified in at least two out of the four replicates. Proteins were defined to be induced only if the relative abundance was more than 1.5-fold greater than un-stimulated cells, and if this increase was statistically significant (p<0.05) across four independent biological replicates. Based on these selection criteria, we identified 16 candidates as nascent proteins that were induced (≧1.5-fold, p<0.05) upon stimulation with either TNF-α or IL-1β or both after 4 hr of cytokine stimulation (Table 1).

TABLE 1

| | TNF-α | | IL-1β | |
|---|---|---|---|---|
| | Fold change | p ≦ | Fold change | p ≦ |
| DDX17—DEAD (Asp-Glu-Ala-Asp) box | 1.85 | 0.03 | 2.2 | 0.001 |
| polypeptide 17 | | | | |
| CAP1—CAP, adenylate cyclase-associated | 1.76 | 0.06 | 1.9 | 0.009 |
| protein 1 | | | | |
| ALDOA—aldolase A, fructose-bisphosphate | 1.59 | 0.04 | 1.5 | 0.05 |
| PYGL—phosphorylase, glycogen, liver | 1.63 | 0.05 | 1.7 | 0.05 |
| CTPS—CTP synthase | 1.58 | 0.05 | 1.5 | 0.06 |
| HMGN1—high-mobility group nucleosome | 1.87 | 0.05 | 1.5 | 0.08 |
| binding domain 1 | | | | |
| EIFA3—eukaryotic translation initiation | 1.59 | 0.04 | 1.2 | 0.09 |
| factor 3, subunit A | | | | |
| PRTN3—proteinase 3 | 1.52 | 0.03 | 0.8 | NS |
| HSPA4—heat shock 70 kDa protein 4 | 1.56 | 0.03 | 1.1 | NS |
| AIFM1—apoptosis-inducing factor, | 1.59 | 0.04 | 1.2 | NS |
| mitochondrion-associated, 1 | | | | |
| CALR—calreticulin | 1.51 | 0.05 | 1.0 | NS |
| VDAC3—voltage-dependent anion channel 3 | 1.51 | 0.05 | 1.1 | NS |
| RAB14—member RAS oncogene family | 1.53 | 0.05 | 1.2 | NS |
| PSME2—proteasome (prosome, macropain) | 2.16 | 0.08 | 2.3 | 0.04 |
| activator subunit 2 (PA28 beta) | | | | |
| SEC22B—vesicle trafficking protein | 1.24 | NS | 1.6 | 0.01 |
| homolog B | | | | |
| LCP1—lymphocyte cytosolic protein 1 (L- | 1.08 | NS | 1.5 | 0.03 |
| plastin) | | | | |

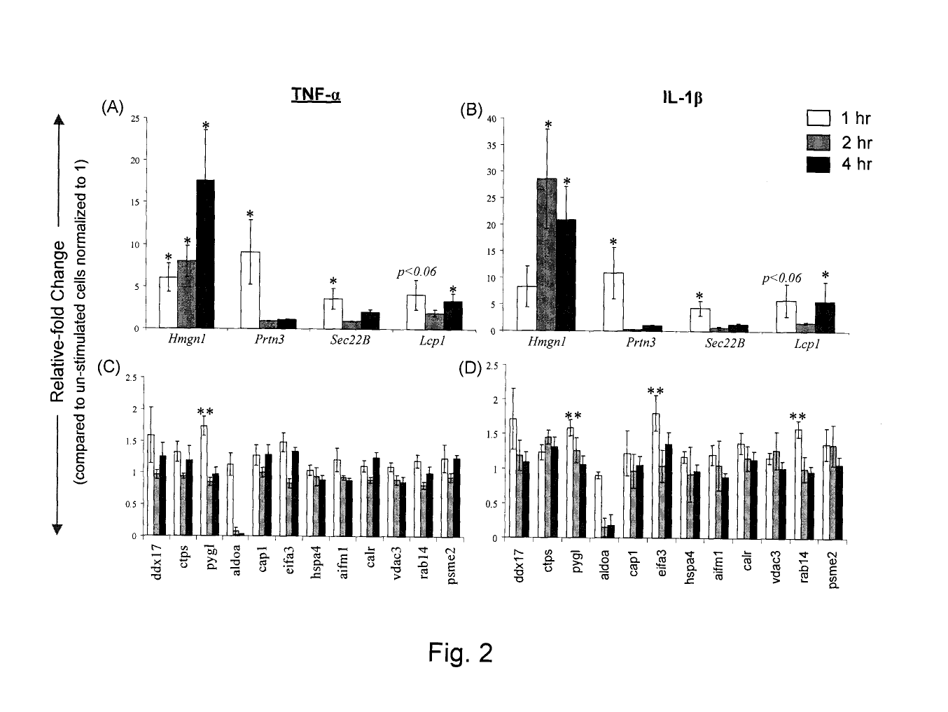

TNF-α and IL-1β induced gene expression of five common identified molecular indicators: In order to confirm that the identified 16 proteins (Table 1) were indeed being induced upon stimulation with the cytokines, we evaluated kinetics of cytokine-induced gene expression for these candidates by quantitative real-time PCR (qRT-PCR). Human THP-1 monocytic cells were stimulated with either TNF-α or IL-1β, and transcriptional responses for the 16 identified candidates (Table 1) were monitored after 1, 2 and 4 hr of cytokine stimulation. Both TNF-α and IL-1β induced gene expression of five of the 16 candidates (FIGS. 2(A)-2(D)). Gene expression of HMGN1, LCP-1, sec22B and prtn3 (C-ANCA), was up-regulated between 4 and 18-fold (p≦0.05), in the presence of either TNF-α or IL-1β after 1 hr of stimulation (FIGS. 2A and 2B). Both TNF-α and IL-1β induced a modest (≧1.5-fold) but significant (p≦0.01) up-regulation of a glycogen phosphorylase enzyme, PYGL (FIGS. 2C and 2D). TNF-α did not uniquely induce the gene expression of any candidate monitored in this study. In contrast, there was a modest (≧1.5-fold, p≦0.01) up-regulation of RAB14 and Eifa3, upon stimulation with IL-1β but not TNF-α (FIG. 2D).

Induction of HMGN1 nascent protein (Table 1) as well as up-regulation of gene expression (FIG. 2) were demonstrated upon stimulation with either TNF-α or IL-1β in this study. HMGN1 is a nucleosome-binding protein and is an emerging factor in transcriptional regulation of proto-oncogenes and tumour suppressor genes by affecting histone modifications. Post-translational modification of HMGN1, which includes phosphorylation and acetylation, affects the interaction of HMGN1 with its chromatin targets and is thought to regulate cellular responses to environmental stimuli. However the role of HMGN1 in inflammation has not yet been defined. This is the first study that demonstrates that HMGN1 is significantly induced upon stimulation with either TNF-α or IL-1β in human monocytic cells.

Nascent protein synthesis of prtn3 (C-ANCA antigen) was demonstrated to be significant (p≦0.05) upon TNF-α stimulation but not with IL-1β stimulation (Table 1). However, upon monitoring kinetics of gene expression of prtn3, we showed significant up-regulation of prtn3 mRNA after 1 hr of stimulation with both TNF-α and IL-1β (FIGS. 2(A) and 2(B)). Even though de novo protein synthesis of LCP-1 and sec22B was significantly demonstrated in the presence of IL-1β but not TNF-α (Table 1), monitoring of gene expression showed that both LCP-1 and sec22B mRNA was up-regulated after 1 hr of stimulation with either TNF-α or IL-1β (FIGS. 2(A and 2(B)). LCP-1, also known as L-plastin, is a leukocyte-specific protein. It has been previously demonstrated that various inflammatory stimuli such as chemokines, bacterial lipopolysaccharide and immune complexes induce phosphorylation of LCP-1 (Shinomiya et al., 1995, Complete primary structure and phosphorylation site of the 65-kDa macrophage protein phosphorylated by stimulation with bacterial lipopolysaccharide. J. Immunol. 154: 3471-3478; Shibata et al., 1993, Characterization of a 64-kd protein phosphorylated during chemotactic activation with IL-8 and fMLP of human polymorphonuclear leukocytes. I. Phosphorylation of a 64-kd protein and other proteins. J. Leukoc. Biol. 54:1-9) which is required for integrin-mediated adhesion of leukocytes. However, association of the vesicular protein sec22B has not been defined with any inflammatory processes to date.

In summary, we have optimized methods using a combination of metabolic labelling and quantitative proteomics, to identify and quantitative nascent proteins in the presence of exogenous stimuli as follows. We used bioorthogonal non-canonical amino acid tagging (BONCAT) with a surrogate for methionine, L-azidohomolanine (AHA), to label cytokine-induced nascent proteins. The AHA-containing nascent proteins were further tagged with alkyne-biotin for enrichment using affinity purification. Subsequently the nascent proteins were quantified using isobaric iTRAQ® reagents and mass spectrometry (MS). We identified 16 nascent proteins that were significantly (p≦0.05) induced upon cytokine stimulation after 4 hr of stimulation. To validate the identified protein candidates we monitored the kinetics of transcriptional responses after 1, 2 and 4 hr of cytokine stimulation employing quantitative real-time PCR (qRT-PCR). Of the 16 candidates monitored by qRT-PCR, there were five common candidates demonstrated to be induced at the transcriptional level upon stimulation with either TNF-α or IL-1β. Four of these candidates showed robust (more than 4-fold up-regulation of gene expression) and significant (p≦5 0.05) response; (i) a nuclesome binding protein HMGN1, (ii) lymphocyte cytosolic protein 1 (LCP-1), (iii) a serine protease, prtn3, also known as C-ANCA antigen, and (iv) a vesicle trafficking protein sec22b, thereby indicating that these proteins may be common molecular indicators of signalling pathways induced by both TNF and IL-1. A glycogen phosphorylase, PYGL, which is an important metabolic enzyme, was modestly (≧1.5-fold, p≦5 0.01) induced by both TNF-α and IL-1β. Gene expression of two molecular candidates, (i) a RAS family oncogene, RAB14, and (ii) Eifa3, were modestly (≧1.5-fold) but significantly (p≦0.01) up-regulated upon stimulation with IL-1β but not TNF-α. This is the first study to employ a combination of BONCAT and iTRAQ proteomics approaches to quantitatively define nascent proteins induced upon stimulation with either TNF-α or IL-1β.

1. A method for detecting and/or monitoring a chronic inflammation condition associated with rheumatoid arthritis, the method comprising the steps of:
collecting a biological sample from a subject;
detecting in the biological sample, one or more of a HMGN1 protein, a LCP-1 protein, a prtn3 protein, a sec22b protein, and a PYGL glycogen phosphorylase protein thereby producing a test result; and
correlating the test result with a control reference standard.

2. A test kit for diagnosing or monitoring a chronic inflammatory condition associated with rheumatoid arthritis in a test sample, wherein the test kit comprises an antibody for complexing with one of a HMGN1 protein, a LCP-1 protein, a prtn3 protein, a sec22b protein, and a PYGL glycogen phosphorylase protein.

3. The test kit according to claim 2, wherein the test kit comprises a first antibody for complexing with one of a HMGN1 protein, a LCP-1 protein, a prtn3 protein, a sec22b protein, and a PYGL glycogen phosphorylase protein, and a second antibody for complexing with another of the HMGN1 protein, the LCP-1 protein, the prtn3 protein, the sec22b protein, and the PYGL glycogen phosphorylase protein.

4. A composition for treatment of a chronic inflammation condition associated with rheumatoid arthritis, the composition comprising a therapeutically effective amount of an exogenous agonist for decreasing cellular production of and/or cellular activation of and/or cellular expression one or more of a HMGN1 protein, a LCP-1 protein, a prtn3 protein, a sec22b protein, and a PYGL glycogen phosphorylase protein.

5. A method for treatment of a chronic inflammation condition associated with rheumatoid arthritis comprising administering to a patient in need thereof a therapeutically effective amount of a composition of claim 4, thereby reducing inflammation.
